(19) United States
(12) Patent Application Publication
(10) Pub. No.: US 2006/0122657 A1
(43) Pub. Date: Jun. 8, 2006

(54) Programmable voltage-waveform-generating battery power source for implantable medical use

(76) Inventors: Jeffrey Deal, Clarence, NY (US); Glenn Thomas, East Amherst, NY (US)

Correspondence Address: Walter W. Duft;Suite 2, 8616 Main Street, Williamsville, NY, 14221, US

(21) Appl. No.: 11/005,305

(22) Filed: Dec. 4, 2004

(51) Int. Cl. A61N 1/362

(52) U.S. Cl. 607/34

(57) ABSTRACT

A programmable voltage-waveform-generating battery power source for implantable medical use enables an implantable device to deliver therapeutic electrical energy with flexible control of voltage amplitude, waveform and timing. The power source includes a high-energy battery system, a waveform control system and a power amplifier that collectively provide the capability to deliver electrical therapy with varied and programmable voltage waveforms, repetition rates and timing intervals that are unachievable with high voltage energy storage capacitors as presently practiced. The high-energy battery system supplies prime power to the power amplifier, the output of which is connected to physiologic electrodes for the purpose of delivering electrical therapy. The waveform control system is programmable and supplies waveform voltage control inputs to the power amplifier.

### CROSS-REFERENCE TO RELATED APPLICATIONS

This application claims benefit of the filing date of U.S. Provisional Application No. 60/______, filed on Nov. 30, 2004.

### BACKGROUND OF THE INVENTION

1. Field of the Invention

The present invention relates to improvements in the performance of implantable defibrillators, ICDs (Implantable Cardioverter-Defibrillators) and other battery powered medical devices designed to provide high-energy electrical stimulation of body tissue for therapeutic purposes.

2. Description of Prior Art

High-energy battery powered medical devices, such as implantable defibrillators and ICDs are designed to deliver a strong electrical shock to the heart when called upon to correct an onset of tachyarrhythmia. In traditional devices the high-energy pulse is produced by charging one or more high-voltage energy storage capacitors from a low-voltage battery and then rapidly discharging the capacitors to deliver the intended therapy. This concept is widely practiced and disclosed in numerous patents, including U.S. Pat. No. 4,475,551 of Mirowski dated Oct. 9, 1984. Additionally, much clinical data on defibrillation therapy has been collected and published. See, for example, Gregory P. Walcott et al., “Mechanisms of Defibrillation for Monophasic and Biphasic Waveforms,” Pacing and Clinical Electrophysiology, March1994:478; and Andrea Natale et al., “Comparison of Biphasic and Monophasic Pulses,” Pacing and Clinical Electrophysiology, July 1995:1354.

In such devices, the energy is first stored in the electric field within one or more capacitors and subsequently transferred to the body tissue. The voltage waveform of the resulting therapy pulse is therefore constrained to consist of one or more truncated exponential decay shapes because of the fact that the capacitors are charged to store only an amount of energy marginally greater than that which is required to be delivered to the body tissue. The capacitor voltage will therefore be a maximum at the start of the discharge pulse and will decay to a lower value at the terminus of the discharge pulse. Likewise, the capacitor must be recharged after delivery of a therapy pulse before a subsequent therapy pulse can be delivered. This fundamental limitation on the voltage waveform of the discharge pulse has a number of serious shortcomings that limit the efficacy of the medical device and contribute to patient discomfort. Chief among these shortcomings are lack of independent control over the voltage, energy and duration of the therapy pulse and a lack of control over the rapidity at which therapy pulses may be delivered.

The energy stored in any capacitor is given by the relationship E=½*C*V², so that the two parameters of energy and voltage are not independently controllable. Thus, one technique that is widely practiced to control the amount of energy to be delivered to the body tissue is by limiting the maximum voltage to which the energy storage capacitors are charged.

It is also well known to those skilled in the art that the therapy regimes for most, if not all, defibrillators and ICDs dictate increasing energy levels for subsequent therapy pulses when multiple closely spaced defibrillation pulses are required. This is because an unsuccessful outcome for the first therapy pulse is often indicative that the pulse did not deliver sufficient energy to exceed the defibrillation threshold and more energy must be delivered on a subsequent pulse to increase the chance of a successful outcome. The requirement therefore is to deliver subsequent therapy shocks of increasing magnitude until a successful outcome is achieved.

If the energy storage capacitors are charged only to the energy level dictated for the first therapy pulse, they must be recharged to a higher energy level after a determination is made that the first pulse had an unsuccessful outcome. Conversely, if the energy storage capacitors are fully charged when the need for therapy is first recognized in anticipation of requiring multiple shocks, the first shock delivered would have the most energy, with any subsequent shocks delivering less energy unless the capacitors are fully recharged between pulses. This protocol thus does not eliminate the need to recharge the capacitors between therapy pulses and in many cases wastes energy by charging the capacitors with energy that is not delivered for therapy. In either case, there is some minimum time delay until the first therapy pulse can be delivered and between the delivery of each subsequent therapy pulse because of the need to recharge the capacitors. There is clinical data (R. Gradhaus et al., “Effect of Ventricular Fibrillation Duration on the Defibrillation Threshold in Humans.” Journal of Pacing and Clinical Electrophysiology, 2001; 25:14-19; and S. Windecker et al., “The Influence of Ventricular Fibrillation Duration on Defibrillation Efficacy Using Biphasic Waveforms in Humans,” Journal of The American College of Cardiology, 1999; 33:33-38) that indicates a need for higher levels of defibrillation energy as the time from fibrillation onset to defibrillation shock increases. A significant time delay to defibrillation therapy is also undesirable because of the increasing risk of tissue damage due to lack of blood perfusion with every second that passes while the heart is not beating.

It is to improvements in the delivery of high-energy therapy that the present invention is concerned. In particular, the invention is directed to the provision of programmable voltage waveforms for therapeutic delivery by an implantable defibrillator, ICD or other battery-powered medical device.

### SUMMARY OF THE INVENTION

It is an object of the invention to provide an implantable medical device that is capable of delivering high-energy electrical therapy with voltage waveforms that are varied and programmable. This implantable medical device is capable of delivering the voltage and energy required for defibrillation of a human heart as well as other modes of therapy requiring less energy.

A further object of the invention is to provide an implantable defibrillator or cardioverter-defibrillator wherein the use of a high-energy/high-voltage battery power source provides for the rapid delivery of defibrillation shocks without the need for delay required to charge high-voltage capacitors.

A further object of the invention is to provide an implantable defibrillator or cardioverter-defibrillator wherein the capability to deliver varied and programmable voltage waveforms provides for improved probability of successful defibrillation with lower levels of delivered energy.

A further object of the invention is to provide an implantable defibrillator or cardioverter-defibrillator wherein the capability to deliver varied and programmable voltage waveforms provides therapy with reduced patient discomfort.

The foregoing objects are achieved and an advance in the art is provided by a programmable voltage-waveform-generating battery power source for implantable medical devices, such as implantable defibrillators and ICDs. The power source includes a high-energy battery system, a waveform control system and a power amplifier that collectively provide the capability to deliver electrical therapy with varied and programmable voltage waveforms. The high-energy battery system supplies prime power to the power amplifier, the output of which is connected to physiologic electrodes for the purpose of delivering electrical therapy. The waveform control system supplies waveform voltage control inputs to the power amplifier.

The high-energy battery system may be constructed with a multiplicity of low-voltage rechargeable cells that are interconnected to provide a medium-to-high voltage source suitable for delivering electrical stimulation therapy to tissue within the human body. For example, the high-energy battery system may utilize rechargeable thin-film lithium cells wherein a multiplicity, e.g. 10-250, of independent cells are fabricated and packaged in a total volume equivalent to the existing energy storage capacitors, i.e. 10 to 20 cm³. The cells are electrically interconnected in either a fixed or dynamically configurable fashion in order to deliver electrical energy at a voltage and current consistent with the maximum requirements for therapy needs to be met by the device in which the power source is implemented. In the case of a defibrillator or ICD, the maximum voltage may be as much as 800 volts at peak currents of 20-30 amperes. For lower energy applications such as muscle or nerve stimulation the maximum voltage and current requirements would be reduced.

The waveform control system has the ability to produce a plurality of waveform control outputs. Each waveform control output corresponds to waveform information stored in a memory of the waveform control system. The waveforms are selectable according to therapeutic requirements. The amplitude of the waveform control output can also be specified to the waveform control system. Waveform slope can also be controlled, and reverse image waveforms can also be generated.

The power amplifier can be implemented using a high-efficiency class D switching mode amplifier that modulates the output of the high-energy battery system according to the waveform control output of the waveform control system. A pulse width/duty cycle control module of the power amplifier is driven by an oscillator to convert the waveform control output into voltage pulses. The voltage pulses are provided to the gate of a field effect transistor whose source is connected to the high-energy battery system and whose drain is connected to a two-pole low-pass output filter. The filter integrates the energy in the voltage pulses over time to provide an amplified output voltage that is proportional to the waveform control output of the waveform control system.

According to one exemplary embodiment of the invention, an implantable defibrillator utilizes an implementation of the inventive power source in which the high-energy battery system provides high-voltage energy to the power amplifier and the latter's output is connected to physiologic electrodes, e.g. a defibrillation catheter. The high-energy battery system is configured such that the individual cells are charged in a parallel circuit arrangement and discharged in a series circuit configuration. The low-voltage recharging energy is provided from a primary cell with high-energy density. These configurations allow recharging at a low voltage potential and discharging at a much higher potential.

According to another exemplary embodiment of the invention, an implantable defibrillator again utilizes an implementation of the inventive power source in which the high-energy battery system provides high-voltage energy to the power amplifier and the latter's output is connected to physiologic electrodes, e.g. a defibrillation catheter. The high-energy battery system is again configured such that the individual cells are charged in a parallel circuit arrangement and discharged in a series circuit configuration. The low voltage recharging energy is provided from a transcutaneous RF induction charging system.

According to yet another exemplary embodiment of the invention, an implantable defibrillator again utilizes an implementation of the inventive power source in which the high-energy battery system provides high-voltage energy to the power amplifier and the latter's output is connected to physiologic electrodes, e.g. a defibrillation catheter. The high-energy battery system comprises a primary or secondary battery assembly that provides high voltage energy to a switching mode amplifier whose output is connected to physiologic electrodes, e.g. a defibrillation catheter. The primary or secondary battery has sufficient total energy to support the total energy requirements of the device throughout the design lifetime of the device.

### BRIEF DESCRIPTION OF THE DRAWINGS

The foregoing and other features and advantages of the invention will be apparent from the following more particular description of exemplary embodiments of the invention, as illustrated in the accompanying Drawings in which:

FIG. 1 is a functional block diagram of an exemplary programmable voltage-waveform-generating battery power source constructed in accordance with the invention;

FIG. 2 is a functional block diagram of an exemplary waveform control system for generating multiple voltage waveforms in the power source of FIG. 1;

FIGS. 3A, 3B and 3C are graphs depicting a series of waveforms that might be generated by the waveform control system depicted in FIG. 2;

FIG. 4 is a simplified schematic diagram of a class D switching mode amplifier for the power source of FIG. 1;

FIG. 5 is a graph depicting a switched supply voltage with applied pulse width modulation and a resulting trapezoidal output voltage waveform of the amplifier of FIG. 4;

FIG. 6 is a functional block diagram of an implantable defibrillator implemented with the power source of FIG. 1, and wherein energy for recharging the high-energy battery system is provided by a primary battery;

FIG. 7 is a functional block diagram of an implantable defibrillator system implemented with the power source of FIG. 1, and wherein energy for recharging the high-energy battery system is provided by a transcutaneous charging system; and

FIG. 8 is a functional block diagram of an implantable defibrillator system implemented with the power source of FIG. 1, and wherein the high-energy battery system is a primary battery system or a secondary battery system with sufficient total energy capacity to satisfy the defibrillation energy requirements for the design lifetime of the device.

### DETAILED DESCRIPTION OF EXEMPLARY EMBODIMENTS

Introduction

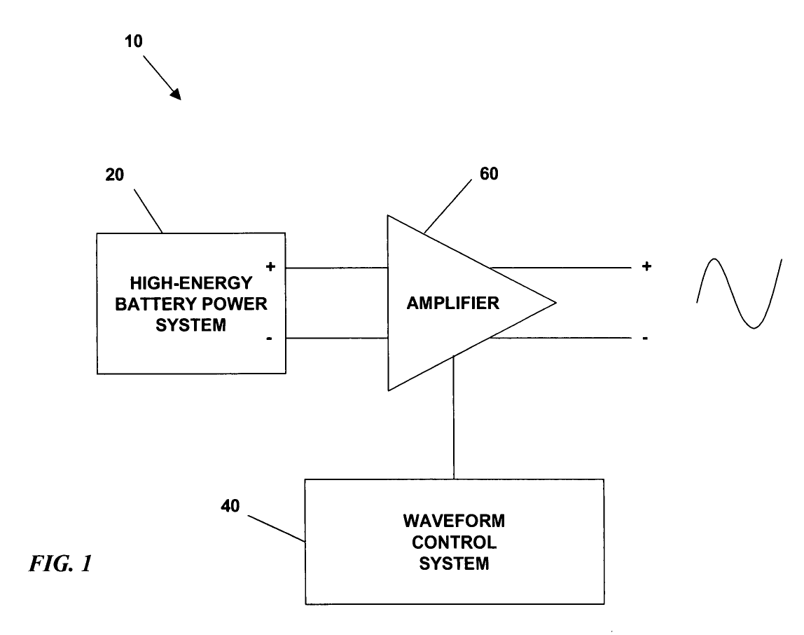

Turning now to the drawings, wherein like reference numerals indicate like elements in all of the several views, FIG. 1 illustrates an exemplary design for a programmable voltage-waveform-generating power source 10 for use with implantable defibrillators, ICDs and other battery powered medical devices. As indicated by way of summary above, the power source 10 can be provided with a high-energy battery system 20, a waveform control system 40 and a power amplifier 60. The high-energy battery system 20 is utilized on an intermittent basis to store and release electrical energy in order to deliver electrical energy to body tissue for therapeutic purposes. The high-energy battery system 20 provides its high-voltage energy output to the power input of the power amplifier 60. The waveform control system 40 provides a control input to the amplifier 60 that controls the amplifier's power output to produce a varied and programmable voltage waveform that is optimized to provide tissue stimulation or therapy based upon predetermined parameters. Each of these components will now be described in more detail.

High-Energy Battery System

The high-energy battery system 20 can be constructed with a multiplicity of low-voltage battery cells that are interconnected to provide a medium-to-high voltage source suitable for delivering electrical stimulation therapy to tissue within the human body. In the case where the power source 10 is implemented in a defibrillator or ICD, the maximum voltage delivered by the high-energy battery system 20 may be as much as 800 volts at peak currents of 20-30 amperes. For lower energy applications such as muscle or nerve stimulation, the maximum voltage and current requirements would be reduced.

By way of example only, the high-energy battery system 20 may utilize rechargeable thin-film lithium cells wherein a multiplicity, e.g. 10-250, of independent cells are fabricated and packaged in a total volume equivalent to the existing energy storage capacitors, i.e. 10 to 20 cm³. Thin-film battery cell construction techniques, such as those disclosed in U.S. Pat. Nos. 6,818,356, 6,517,968, 5,597,660, 5,569,520, 5,512,147 and 5,338,625, and in published application US2004/0018424, can be used to fabricate such cells. The contents of the foregoing patents and patent applications are hereby incorporated herein by this reference.

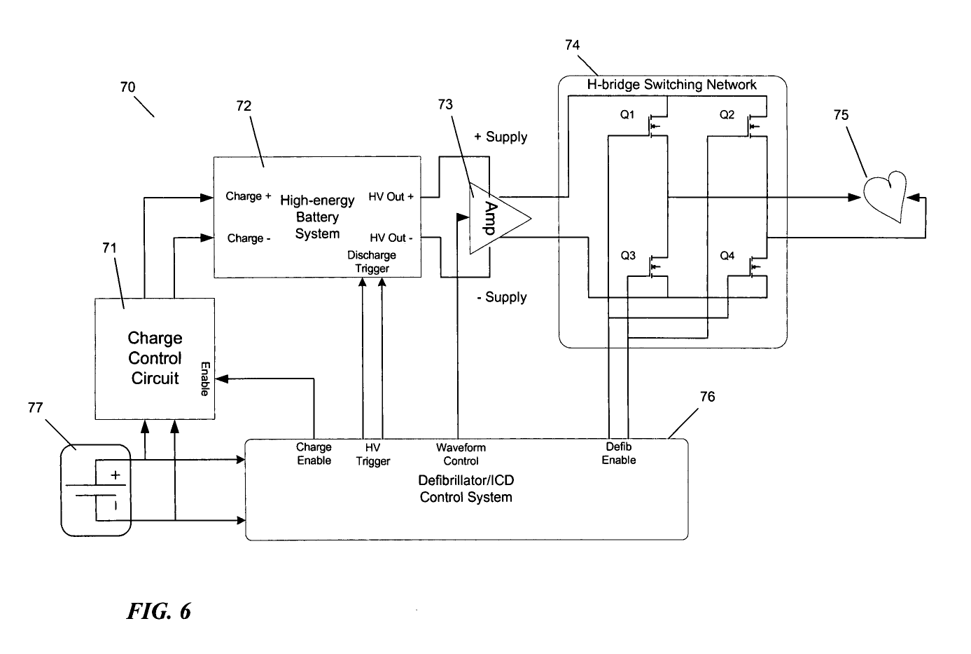

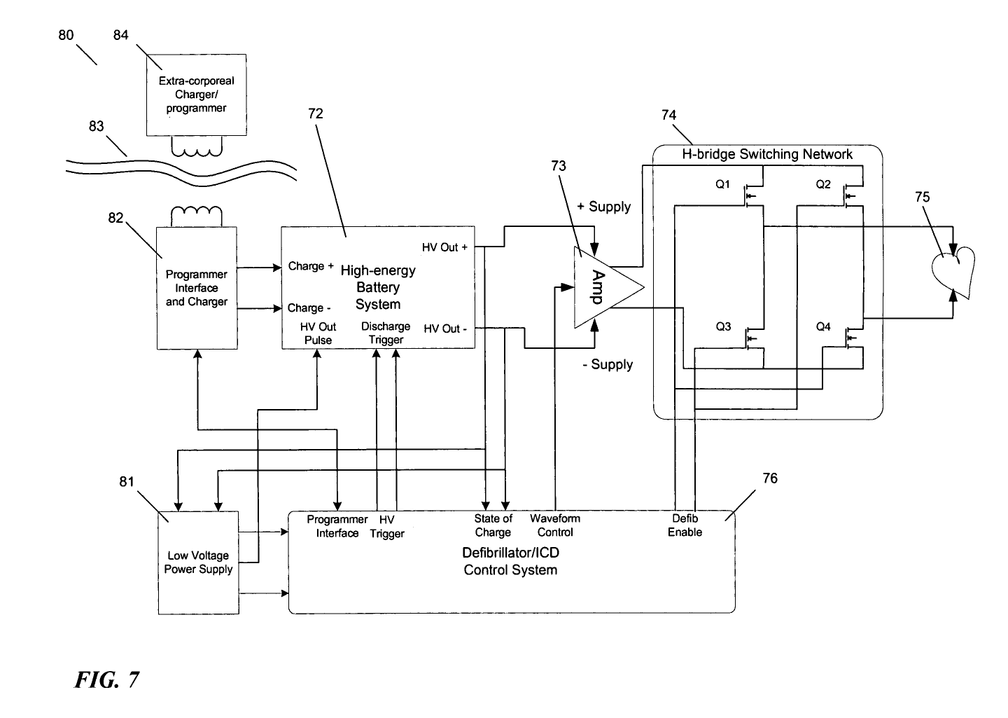

The multiplicity of cells of the high-energy battery system 20 can be electrically interconnected in either a fixed or dynamically configurable fashion in order to deliver electrical energy at a voltage and current consistent with the maximum requirements for therapy needs to be met by the device in which the power source is implemented. One exemplary connection configuration that may be used to electrically interconnect the multiplicity of cells is disclosed in U.S. Pat. No. 5,369,351. Another exemplary connection configuration is disclosed in commonly-assigned copending application Ser. No. 10/994,565, filed on Nov. 22, 2004 by Wilson Greatbatch et al. for a “High Energy Battery Power Source For Implantable Medical Use.” The contents of the foregoing patent and patent application are hereby incorporated herein by this reference. Additional design options for the high-energy battery system 20 are discussed in more detail below in connection with FIGS. 6-8.

Waveform Control System

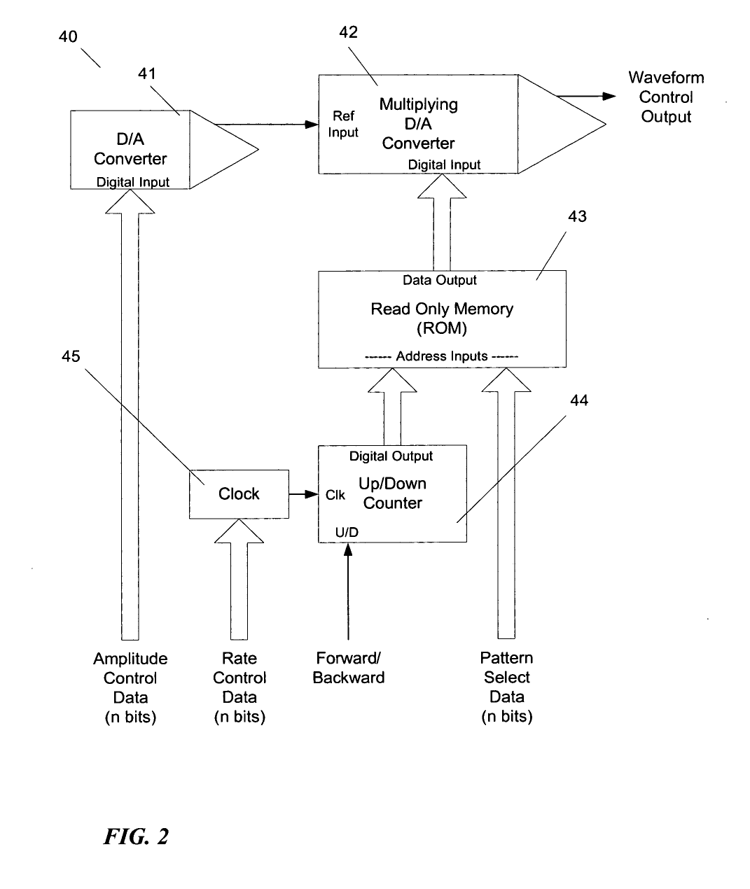

Turning now to FIG. 2, an exemplary construction of a waveform control system 40 is shown that can generate and vary multiple voltage waveforms. A first digital-to-analog (D/A) converter 41 is provided to accept a digital data input (labeled “Amplitude Control Data”) that represents the maximum amplitude of the output waveform to be generated. This digital data is provided as one control output from a microprocessor control system (not shown) that would be integral to an implantable device in which the power source 10 is implemented. The output of the D/A converter 41 is an analog voltage or current proportional to the desired maximum waveform amplitude. This analog parameter is applied to the reference input of a multiplying D/A converter 42. The digital data input to this second D/A converter 42 is provided by the data outputs of a read-only memory (ROM) 43. The multiplying D/A converter 42 produces an analog voltage or current output that is the product of the reference input multiplied by the magnitude of the digital number applied to the digital input. The output labeled “Waveform Control Output” is therefore representative of the product of the reference input and digital data inputs to the multiplying D/A converter 42.

The digital patterns of data necessary to construct the desired output waveforms are stored within the ROM 43. The data patterns are developed during design of the device and hard coded into the ROM 43, and the total number of patterns that may be generated is limited only by the address space of the ROM 43. Each pattern may be stored in a separate area of the ROM 43 address space and selected by a subset of the address inputs. This selection data is provided from the integral microprocessor system of the device in which the power source 10 is implemented as the digital data labeled “Pattern Select Bits.” If the data is provided in binary format, three bits of data would allow for the selection of 2³=8 different waveform patterns, e.g. rectangular, trapezoidal, triangular, Gaussian, sinusoidal. The generation of the patterns is accomplished by sequentially stepping through the ROM 43 address space. Discrete values representative of a piecewise approximation of each waveform are predefined and stored in the ROM 43 at the time of fabrication. The digital outputs of a binary up/down counter 44 are applied to the remaining address inputs of the ROM 43 so that as the counter is incremented or decremented, the predefined digital values representative of the waveform amplitude will be sequentially selected and applied to the digital input of the multiplying D/A converter 42. The up/down counter 44 is capable of incrementing or decrementing as selected by the input labeled “Forward/Backward,” which is another output from the integral microprocessor system of the device in which the power source 10 is implemented. This control input provides for the capability to generate each stored waveform or its mirror image without utilizing additional address space within the ROM 43.

Finally, the rate of change and time duration of each waveform is controlled by the rate at which the up/down counter 44 is incremented or decremented. A variable frequency clock 45 has its output connected to the clock input of the up/down counter 44. The clock frequency is controlled by a digital input value labeled “Rate Control Data” which is also supplied as a control output of the integral microprocessor system of the device.

In summary, the waveform control system 40 depicted in FIG. 2 provides a means by which multiple analog waveforms may be generated under control of a microprocessor or similar digital control system. The waveform control system 40 includes digital inputs to select from a multiplicity of available waveforms and digital controls for waveform amplitude and rate.

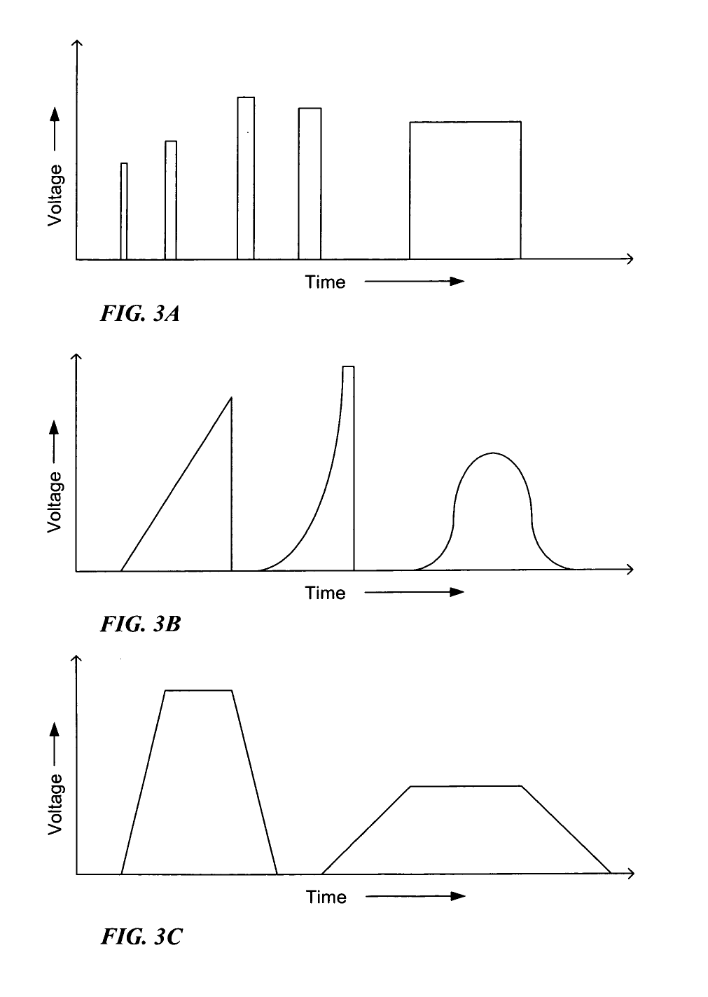

Turning now to FIG. 3A, 3B and 3C, three graphs of exemplary waveform voltage vs. time are shown to illustrate the capabilities of the waveform control system 40 depicted in FIG. 2. FIG. 3A shows a number of rectangular pulses of varying amplitude and duration. FIG. 3B shows an ascending triangle followed by descending triangle. A person skilled in the art would recognize that both these waveforms can be generated with the same data set in the waveform control system 40 depicted in FIG. 2 by incrementing and decrementing the up/down counter 44 and changing the rate at which the counter is clocked. FIG. 2C shows two different trapezoidal waveforms. Again, the same data set could be used to create both waveforms by simply altering the clocking rate. The maximum amplitude may be controlled as previously explained.

Power Amplifier

Turning now to FIG. 4, the power amplifier 60 is shown by way of a simplified schematic diagram in which the power amplifier is implemented as a class D switching mode amplifier. Direct current prime power is provided to the input circuits labeled “+Supply” and “−Supply.” A capacitor 61 is provided on the prime power input for high-frequency AC decoupling. A constant frequency oscillator 62 provides a switching input to the circuitry 63 labeled “Pulse Width/Duty Cycle Control.” While the oscillator symbol in FIG. 4 depicts a sinusoidal waveform, persons skilled in the art will understand that a trapezoidal switching waveform is more commonly used in practice. The circuitry 63 may be powered by the prime power input, as shown, or by a lower voltage prime power source (not shown). The decoupled “+Supply” circuit is connected to the source terminal of a p-channel enhancement mode metal oxide semiconductor field effect transistor (MOSFET) 64. The drain terminal of the MOSFET 64 is connected to one terminal of an inductor 66 and a catch diode 65. The second inductor terminal is connected to one terminal of an output filter capacitor 67. The other terminal of the output filter capacitor 67 is returned to the common circuit labeled “−Supply” and “−Output.” The gate terminal of the MOSFET 64 is connected to the output of the circuitry 63 labeled “Pulse Width/Duty Cycle Control.” The control circuit 63 has a single input labeled “Control Input.”

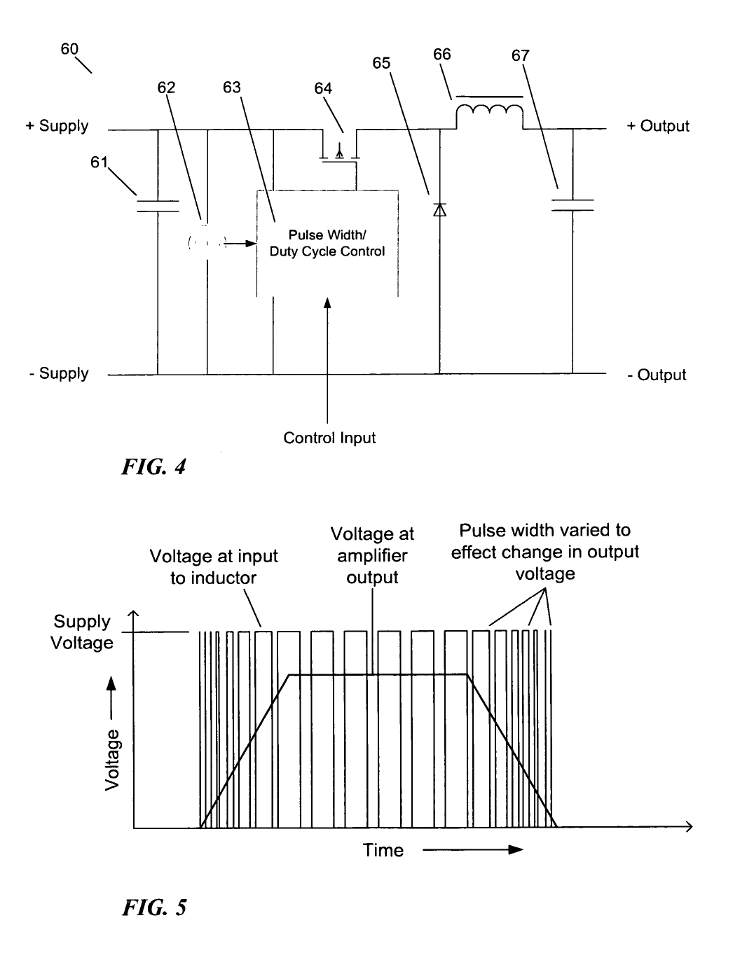

The operation of the class D amplifier is depicted graphically in FIG. 5 and explained here. When the signal labeled “Control Input” is at a quiescent or zero value, the MOSFET 64 is held in a non-conducting state by driving the gate terminal voltage to a value equal to the source terminal voltage. The drain terminal voltage will be zero if the MOSFET 64 is not conducting. As the voltage on the “Control Input” rises to a non-zero value, the “Pulse Width/Duty Cycle Control” circuitry 63 will apply negative voltage pulses of varying width to the gate terminal of the MOSFET 64 and cause the transistor to conduct energy in short bursts. Referring now to FIG. 4, a graph of voltage vs. time is shown with two variables plotted. The series of rectangular pulses of constant height and varying width depict the voltage on the drain terminal of the MOSFET 64 which is connected to the input of the filter inductor 66. A catch diode 65 is provided at the input to the inductor 66 so that the when the MOSFET 64 turns off and the magnetic field in the inductor collapses, the inductor current will flow through the diode and into the output filter capacitor 67. Superimposed underneath these pulses is a single trapezoidal waveform that depicts the resulting output voltage on the circuit labeled “+Output.” It is important to note that the drain voltage of the MOSFET 64 toggles between two discrete values of zero volts and the maximum voltage which is essentially equal to “+Supply.” In a class D configuration the switching element (MOSFET 64) is operated as a saturated switch so that the transistor either withstands maximum drain-source voltage with minimum drain-source current or minimum drain-source voltage with maximum drain-source current. This mode of operation minimizes the power dissipated in the switching element and provides a very high efficiency amplifier with exceptionally low losses.

The drain voltage of the MOSFET 64 depicted in FIG. 5 consists of a series of discrete voltage pulses whose pulse widths are directly proportional to the desired output voltage. A two-pole low-pass output filter is provided by inductor 66 and capacitor 67. These two elements integrate the energy in the voltage pulses over time to remove the switching frequency and harmonics, thereby providing an output voltage on the “+Output” circuit which is proportional to the voltage on the “Control Input” circuit, but greatly increased in amplitude. Only one configuration of a class D amplifier topology is shown here. A more detailed treatment of class D switching amplifiers is provided in the reference Leach Jr., W. Marshall, “Introduction to Electroacoustics and Audio Amplifier Design, Second Edition—Revised Printing.” Kendall/Hunt, 2001.

Exemplary Implantable Devices

Turning now to FIG. 6, an exemplary implantable device 70 using the concepts taught herein is shown. A microprocessor or other digital control system 76 is integral to the device 70 and controls the operation of all device functions. A high-energy battery power system 72 is provided to supply prime power to a power amplifier 73. The high-energy battery power system 72 corresponds to the high-energy battery power system 20 described above, and is constructed with a multiplicity of low-voltage (e.g., 3-4 volts) rechargeable batteries, such as thin-film lithium cells, suitably connected to facilitate charging in parallel and discharging in serial at high voltage (e.g., 120-800 volts). The power amplifier 73 corresponds to the above-described power amplifier 60. Note however, that although the latter was described as implementing a class D amplifier topology, it should be understood that other amplifier topologies may be used to achieve the same results. The outputs of the power amplifier 73 are supplied as inputs to a conventional H-bridge switching network 74 of the type that is well-known to those skilled in the art. The outputs of the H-bridge switching network 74 are connected to a defibrillation catheter or other physiologic electrodes for the purpose of delivering therapeutic electrical stimulation to a heart 75 or other tissue. Primary energy for the implantable device 70 is delivered by a high-energy density primary battery 77. This battery 77 provides prime power for the device control system 76 and also supplies energy to a charge control circuit 71. The device control system 76 incorporates a waveform control system whose purpose is to provide waveform control inputs to the power amplifier 73, as described above in connection with the waveform control system 40. The purpose of the charge control circuit 71 is to regulate the flow of energy from the primary battery 77 to the rechargeable high-energy battery system 72 when recharging is required.

The operation of the implantable device 70 will now be described. During periods of normal syncope in the heart 75, or when very low energy pacing is required, the components of the high-energy system will be dormant. Low level activity will be supported by the primary battery 77 and circuitry within the device control system 76 that is not shown here. At such time that the heart enters an abnormal condition such as tachycardia or fibrillation when higher energy therapy is required, the device control system 76 will detect the need for therapy and select a therapy waveform based upon predetermined thresholds and parameters. The device control system 76 will enable the high-energy battery system 72 by asserting the signals applied to the inputs labeled “Discharge Trigger.” The high-energy battery system 72 will provide high voltage energy to the prime power inputs of the power amplifier 73 that are labeled “+Supply” and “−Supply.” The device control system 76, and particularly the waveform control circuitry therein, will then produce a low amplitude therapy waveform on the output labeled “Waveform Control,” which is supplied as the control input to the power amplifier 73. The power amplifier 73 will reproduce the waveform at a higher power level and supply it to the H-bridge switching network 74. The device control system 76 will simultaneously enable the outputs labeled “Defib Enable” singly or in sequence to cause the H-bridge switching network 74 to connect the power amplifier 73 outputs to the physiologic electrodes. The polarity of the output energy is determined by which of the two “Defib Enable” outputs is enabled by the device control system 76 at any time during any waveform. By this means, the device control system 76 may select a monophasic or biphasic output waveforms depending upon the therapy requirements. In the event that the high-energy battery system 72 requires recharging, the control system 76 will assert the output labeled “Charge Enable” that is supplied as an input to the charge control circuit 71. When this circuit is enabled the charge control circuit 71 will transfer energy from the primary battery 77 to the high-energy battery system 72 to recharge it.

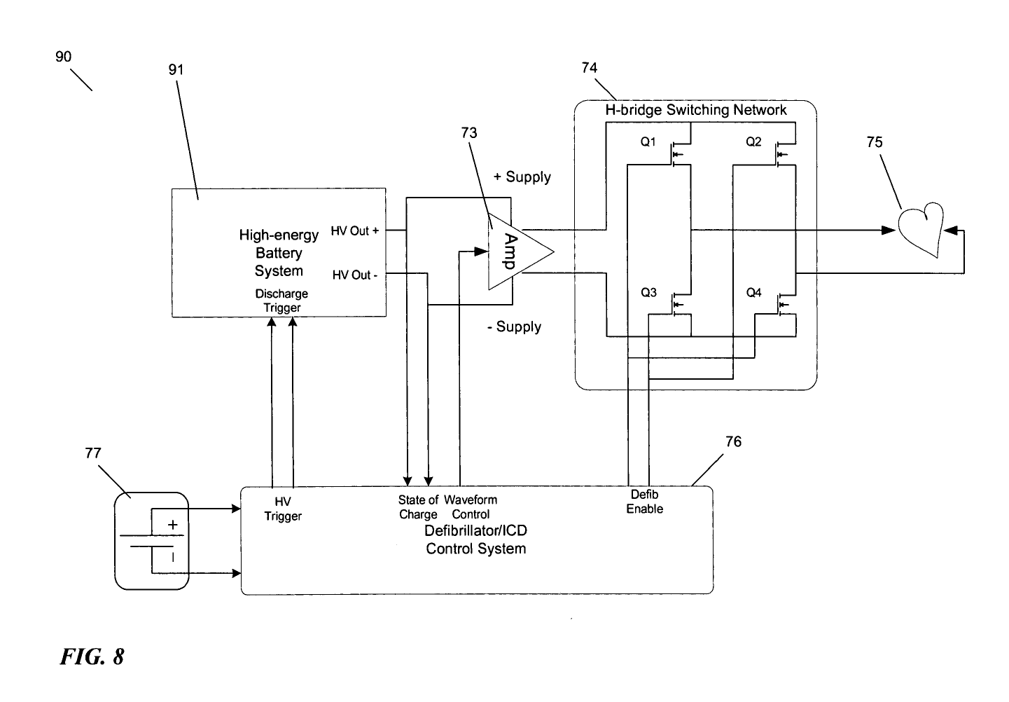

A second exemplary implantable medical device 80 is depicted in FIG. 7. This device is similar to the implantable device 70 disclosed in FIG. 6 (as shown by the use of corresponding reference numerals) with the exception that no primary battery is provided. Instead, all energy for the operation of the device is obtained from the high-energy battery system 72. The high-voltage output of the high-energy battery system 72 is supplied to a low-voltage power supply 81 that provides the low voltage/low power needed by the device control system 76. This voltage is typically 2-3 volts at a power level of 20-50 microwatts. If the low-voltage power supply 81 is implemented with a charge pump topology, the circuit will enable the high-voltage output of the high-energy battery system 72 for very short periods by asserting the signal connected to the input labeled “HV Out Pulse.”

The high-energy battery system 72 is provided with sufficient energy storage capability to provide all required device and therapy power for many months of operation. On a yearly basis or at some other suitable interval, the patient will be required to visit a doctor for a checkup and recharging of the high-energy battery system 72. The doctor will use an extra-corporeal charger/programmer 84 to communicate with the implantable device 80 and to transmit energy to the device for the purpose of recharging the high-energy battery system 72. This charger/programmer 84 conventionally utilizes low frequency/low power RF energy to transmit energy through the patient's skin 83.

Turning finally to FIG. 8, a third exemplary implantable medical device 90 is shown. Again, as shown by the use of corresponding reference numerals, the implantable device 90 is similar to the implantable device 70 of FIG. 6, and operates in the same fashion when delivering high-energy therapy or stimulation. In the implantable device 90, however, the high-energy battery system 91 is a primary or secondary battery with sufficient energy capacity to deliver the total required therapy energy throughout the device service lifetime. The low voltage/low energy prime power requirements for the control system 76 are met by a primary battery 77. No recharging system is required.

Rationale for Configuration

Most, if not all implantable defibrillators and cardioverter-defibrillators utilize high voltage energy storage capacitors as the means to accumulate an electrical charge and then deliver that charge to the heart tissue in order to simultaneously depolarize enough of the heart cells to stop fibrillation and allow the heart to resume normal sinus rhythm. The selection of high-voltage capacitors for defibrillators came about because of the need to deliver significant amounts of energy in a short period of time. By way of example, most modern ICDs are capable of delivering shocks with a total energy of 30 joules with shock durations of less than 50 milliseconds. No other energy delivery/storage technology has been known or practiced with the capability to store and rapidly deliver this level of energy in a small volume consistent with the requirements for an implantable device.

There is a fundamental shortcoming to defibrillators and ICDs that arises from the use of high-voltage capacitors for energy storage. The heart tissue and surrounding blood are electrically coupled to the defibrillator or ICD by means of physiologic electrodes, and the nature of the tissue is such that a relatively low impedance load is presented to the output of the defibrillator. This load is primarily resistive in nature, and endocardial defibrillation catheters as presently practiced typically present an impedance with respect to the device enclosure on the order of 40 ohms. When defibrillation is required, the high-voltage capacitors are charged to as much as 800 volts and then connected to the catheter/device enclosure to deliver the stored energy to the heart. The resulting voltage/current waveform is a decaying exponential waveform with the highest voltage occurring on the leading edge of the waveform. The first generation of implantable defibrillators provided a single monophasic discharge pulse to achieve defibrillation. Subsequent clinical studies revealed that a higher probability of successful defibrillation could be achieved with lower total energy levels by using a biphasic discharge waveform. The biphasic waveform is typically achieved by interrupting the discharge circuit when roughly 50% of the capacitor energy has been delivered and reversing the polarity of the connection to deliver the remaining stored energy. The significant differences between monophasic and biphasic waveforms are discussed in detail in G. Walcott et al., “Mechanisms of Defibrillation for Monophasic and Biphasic Waveforms”, Journal of Pacing and Clinical Electrophysiology, 18, 478-498, (1994); and A. Natale et al., “Comparison of Biphasic and Monophasic Pulses: Does the Advantage of Biphasic Shocks Depend on Waveshape?”, Journal of Pacing and Clinical Electrophysiology, 19, 1354-1361, (1995). As a result of the extensive research and demonstrated advantages of biphasic waveforms, most modern implantable defibrillators and ICDs deliver biphasic defibrillation shocks.

From a clinical perspective, the ultimate requirement is to achieve successful defibrillation with the lowest level of delivered energy. This is desirable for a number of reasons. From a physiologic perspective, higher shock voltages are required for higher energy, and higher shock voltages (approaching 1000 volts) have been found to cause tissue damage. From the perspective of patient comfort, increased shock voltages cause increased levels of patient pain during defibrillation, leading to increased patient anxiety. One means of providing low-pain defibrillation is proposed in U.S. Pat. No. 6,772,007 of Kroll. In this prior art, the inventor disclosed a method of reducing the peak shock voltage while delivering the same energy by means of multiple energy storage capacitors, switches and a current limiting resistor. While this method claims to provide for successful defibrillation at lower peak defibrillation voltages, it lacks flexibility in waveform control, requires a substantial number of components above and beyond a traditional defibrillator circuit and discards some portion of the stored energy in the current limiting resistor. Finally, achieving successful defibrillation at the lowest possible energy is advantageous to the defibrillator or ICD because the device has a fixed maximum amount of energy available for therapy and device background loads. Decreasing the amount of energy required for each defibrillation reduces the drain on the primary battery and thus provides longer device life.

While the available electronic component technology (until very recently) has dictated the use of capacitive discharge circuits for all implantable defibrillators and most, if not all commercially available external defibrillators, there has been within the medical profession long standing interest in improving defibrillation outcome by means of alternative voltage waveforms. External defibrillation with rectangular, trapezoidal and triangular voltage waveforms was studied on animals at various energy levels in an attempt to identify the most efficacious conditions for successful defibrillation. The results of one study were published by Schuder et al.,“Transthoracic ventricular defibrillation with triangular and trapezoidal waveforms”, Circulation Research, Oct. 1966:689-694. A more generalized analysis of defibrillation physiology is provided by W. Irnich, “The Fundamental Law of Electrostimulation and its Application to Defibrillation”, Journal of Pacing and Clinical Electrophysiology;13:1433-1447. In this article, the author asserts that the lowest defibrillation energy is achieved with a rectangular pulse. More recent studies have been published by B. G. Cleland, “A Conceptual Basis for Defibrillation Waveforms”, PACE; 19: 1186-1195 and R. D. White, “Waveforms for Defibrillation and Cardioversion: Recent Experimental and Clinical Studies”, Current Opinion in Critical Care 10:202-207.

We teach here the combination of a high-energy battery system, a waveform control system and a power amplifier within an implantable medical device to provide the capability to deliver varied and programmable voltage waveforms for the purpose of electrostimulation of tissue, including cardiac defibrillation. A device constructed in accordance with this invention will be capable of delivering therapy rapidly, without the many limitations due to energy storage capacitors, over a continuous range of voltages and energy levels not possible with present devices.

Accordingly, a programmable voltage-waveform-generating battery power source for implantable medical use has been disclosed, and the objects of the invention have been achieved. It will, of course, be appreciated that the description and the drawings herein are merely illustrative, and it will be apparent that the various modifications, combinations and changes can be made of these structures disclosed in accordance with the invention. It should be understood, therefore, that the invention is not to be in any way limited except in accordance with the spirit of the appended claims and their equivalents.

We claim:

1. A programmable voltage-waveform-generating battery power source for implantable medical devices that provides the capability to deliver electrical therapy with varied and programmable voltage waveforms, comprising:
a high-energy battery system;
a waveform control system;
a power amplifier;
said high-energy battery system supplying prime power to said power amplifier;
said power amplifier having an output connected to physiologic electrodes for the purpose of delivering electrical therapy; and
said waveform control system supplying a waveform voltage control input to said power amplifier.

2. A power source in accordance with claim 1, wherein said high-energy battery system comprises a multiplicity of rechargeable battery cells electrically connected for charging in parallel and discharging in series.

3. A power source in accordance with claim 2, wherein said rechargeable battery cells comprise a thin-film construction.

4. A power source in accordance with claim 1, wherein said high-energy battery system comprises one of a primary or secondary battery.

5. A power source in accordance with claim 1, wherein said waveform control system comprises a memory for storing information corresponding to a plurality of waveforms.

6. A power source in accordance with claim 1, wherein said waveform control system comprises an input adapted to allow selection of a plurality of waveforms.

7. A power source in accordance with claim 1, wherein said waveform control system comprises an input adapted to allow selection of a waveform amplitude.

8. A power source in accordance with claim 1, wherein said waveform control system comprises an input adapted to allow selection of a waveform slope.

9. A power source in accordance with claim 1, wherein said waveform control system comprises an input adapted to allow selection of a waveform and a reverse image of said waveform.

10. A power source in accordance with claim 1, wherein said power amplifier comprises a class D switching mode amplifier.

11. A power source in accordance with claim 1, wherein said power amplifier comprises a pulse width/duty cycle control module.

12. A power source in accordance with claim 11, wherein said power amplifier comprises an oscillator adapted to drive said pulse width/duty cycle control module to convert said waveform control input into voltage pulses.

13. A power source in accordance with claim 12, wherein said power amplifier comprises a field effect transistor having a gate controlled by said voltage pulses, a source connected to said high-energy battery power system and a drain connected to a two-pole low-pass filter of said power amplifier.

14. A power source in accordance with claim 13, wherein said filter is adapted to integrate the energy of said voltage pulses over time to provide an amplified output voltage that is proportional to said waveform control input.

15. An implantable device comprising:
a high-energy battery system;
a waveform control system;
a power amplifier;
physiologic electrodes;
said high-energy battery system supplying prime power to said power amplifier;
said power amplifier having an output connected to said physiologic electrodes for the purpose of delivering electrical therapy; and
said waveform control system supplying a waveform voltage control input to said power amplifier.

16. An implantable device in accordance with claim 15, wherein said high-energy battery system comprises a multiplicity of rechargeable battery cells electrically connected for charging in parallel and discharging in series, and wherein said implantable device comprises a primary battery adapted to deliver charging energy to said high-energy battery system.

17. An implantable device in accordance with claim 15, wherein said high-energy battery system comprises a multiplicity of rechargeable battery cells electrically connected for charging in parallel and discharging in series, and wherein said implantable device comprises a transcutaneous RF induction charging system adapted to deliver charging energy to said high-energy battery system.

18. An implantable device in accordance with claim 15, wherein said high-energy battery system comprises a primary battery.

19. An implantable device in accordance with claim 15, wherein said high-energy battery system comprises a secondary battery.

20. A programmable voltage-waveform-generating battery power source for implantable medical devices that provides the capability to deliver electrical therapy with varied and programmable voltage waveforms, comprising:
a high-energy battery system;
a waveform control system;
a power amplifier;
said high-energy battery system supplying prime power to said power amplifier;
said power amplifier having an output connected to physiologic electrodes for the purpose of delivering electrical therapy;
said waveform control system supplying a waveform voltage control input to said power amplifier;
said high-energy battery system comprising one of a multiplicity of rechargeable battery cells electrically connected for charging in parallel and discharging in series, or a primary or secondary battery;
said waveform control system comprising a memory for storing information corresponding to a plurality of waveforms, a first input adapted to allow selection of a plurality of waveforms, a second input adapted to allow selection of a waveform amplitude, a third input adapted to allow selection of a waveform slope, and a fourth input adapted to allow selection of a waveform and a reverse image of said waveform;
said power amplifier comprising a class D switching mode amplifier having a pulse width/duty cycle control module, an oscillator adapted to drive said pulse width/duty cycle control module to convert said waveform control input into voltage pulses, a field effect transistor having a gate controlled by said voltage pulses, a source connected to said high-energy battery power system and a drain connected to a two-pole low-pass filter of said power amplifier, and said filter being adapted to integrate the energy of said voltage pulses over time to provide an amplified output voltage that is proportional to said waveform control input.
